(19) United States
(12) Patent Application Publication
(10) Pub. No.: US 2006/0153787 A1
(43) Pub. Date: Jul. 13, 2006

(54) Whitening cosmetics

(75) Inventors: Tsuneo Shinpou, Tokyo (JP); Atsushi Nakajima, Tokyo (JP); Tomohiko Sano, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KAO CORPORATION, Tokyo (JP)

(21) Appl. No.: 11/370,854

(22) Filed: Mar. 9, 2006

Related U.S. Application Data
(63) Continuation of application No. 10/621,335, filed on Jul. 18, 2003.

(30) Foreign Application Priority Data
Jul. 24, 2002 (JP) 2002-215132
Jul. 24, 2002 (JP) 2002-215133

(51) Int. Cl. A61K 36/28

(52) U.S. Cl. 424/62; 424/764

(57) ABSTRACT

Whitening cosmetics contain (A) an oil-soluble chamomilla extract and (B) at least one oil selected from esters of carboxylic acids having 2 to 22 carbon atoms with alcohols having 1 to 22 carbon atoms and vegetable oils. These whitening cosmetics provide good feeling of use upon application, and moreover, are excellent in skin-whitening effect and can satisfactorily prevent formation of spots and freckles which result from sunburn.

### TECHNICAL FIELD

This invention relates to cosmetics excellent in skin-whitening effect.

### BACKGROUND ART

Spots and freckles on the skin generally on excessive deposition of melanine pigment in melanocytes in the skin occur, where the melanocytes have been activated by stimulation through exposure to sunlight, a hormone disorder or a genetic factor and the resulting

With a view to avoiding such spots and freckles, whitening cosmetics have been used conventionally. These whitening cosmetics contain ingredients having a melanogenesis-inhibiting effect, such as L-ascorbic acid and derivatives thereof, hydroquinone derivatives, kojic acid and derivatives thereof, and placenta extracts. These substances, however, show only a feeble melanogenesis-inhibiting effect, failing to provide a sufficient whitening effect.

In recent years, a chamomilla extract is added in numerous cosmetics as an ingredient having an excellent whitening effect. With such cosmetics containing the extract added therein, good feeling of use is hardly available upon application. In addition, depending on the composition, the whitening effect of the chamomilla extract may not be fully drawn out. Therefore the improvement of these problems is desired.

An object of the present invention is, to provide a cosmetic, which provides good feeling of use and moreover, is excellent in whitening effect.

### DISCLOSURE OF THE INVENTION

The inventor has found that cosmetics comprising, an oil-soluble chamomilla extract with an ingredient selected from specific esters and vegetable oils can provide good feeling of use upon application and moreover, an excellent whitening effect.

According to one aspect of the present invention, there is thus provided a whitening cosmetic comprising the following ingredients (A) and (B):
(A) an oil-soluble chamomilla extract, and
(B) at least one oil selected from esters of carboxylic acids having 2 to 22 carbon atoms with alcohols having 1 to 22 carbon atoms and vegetable oils.

### BEST MODE FOR CARRYING OUT THE INVENTION

The oil-soluble chamomilla extract employed as the ingredient (A) in the present invention is obtained by extracting it with a lipophilic organic solvent from flowers of Matricaria chamomilla L. (Compositae). The solvent in the extraction preferably is an oil having the solubility parameter (SP value) in a range of from 15 to 21. Examples of such oil include isopropyl myristate (SP value: 17.0), neopentyl glycol dicaprate (SP value: 17.7), liquid paraffin (SP value: 16.4), and squalane (SP value: 16.2), and mixtures thereof. Further, castor oil, persic oil, soybean oil, sunflower oil and the like can also be available. The term “SP value” as used herein means an index of compatibility between substances, which can be determined by calculating the Hansen's three-dimensional solubility parameter by the method disclosed in JP 3113844 B.

As an extraction method, an oil is added to ground dry chamomilla flowers in an amount 1 to 100 times by weight as much as the chamomilla flowers, followed by extraction under stirring at 10 to 90° C. for 1 to 96 hours. The temperature is appropriately adjusted depending on the oil, which is used.

In general, the kinds and contents of the components contained in the extract vary depending on the oil employed in extraction. In the present invention, an extract obtained using squalane is preferred as it gives a particularly excellent whitening effect.

Such oil-soluble chamomilla extract includes inter alia camazulene, umbelliferone, 7-methoxycoumarine, matricin, matricarin, taraxasterol, upeol, apiin, and a spiro ether compound represented by the following formula (1):

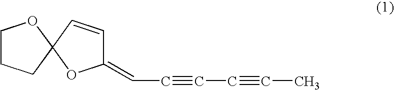

(including both the Z-isomer and E-isomer)

Among these components, especially the content of the spiro ether compound is considered to affect the whitening effect. In the present invention, the content of the spiro ether compound in the oil-soluble chamomilla extract preferably ranges from 10 to 500 ppm. Further, one or two or more of the oil-soluble chamomilla extracts can be used, and in this case, the total content of the spiro ether compound in all the extracts ranges preferably from 10 to 500 ppm.

The content of (A) of the present invention ranges preferably from 0.0001 to 50 wt. %, more preferably from 0.001 to 30 wt. %, particular preferably from 0.005 to 20 wt. % by weight of the cosmetics in terms of the extract because a sufficient whitening effect is available. This content is equivalent to 0.00001 to 5 wt. % in terms of a dry solid content of the extract.

On the other hand, the content of the spiro ether compound in the cosmetic preferably ranges from 0.1 to 50 ppm.

Preferred esters of the carboxylic acids having 2 to 22 carbon atoms with the alcohols having 1 to 22 carbon atoms of (B) can be ester oils having a total carbon number of from 7 to 500. The ester-forming carboxylic acids can be fatty acids (monocarboxylic acids) having a linear or branched, saturated or unsaturated hydrocarbon group of 2 to 22 carbon atoms, preferably 4 to 22 carbon atoms and dicarboxylic acids containing two or more carboxyl groups per molecule and having 2 to 22 carbon atoms, preferably 2 to 8 carbon atoms. On the other hand, the ester-forming alcohols are alcohols having 1 to 22 carbon atoms and can include monohydric alcohols and polyhydric alcohols such as dihydric alcohols and trihydric alcohols. Preferred examples can include monohydric alcohols having 2 to 22 carbon atoms, dihydric alcohols having 2 to 6 carbon atoms, trihydric alcohols having 3 to 6 carbon atoms, and tetrahydric and higher alcohols having 6 to 22 carbon atoms.

Examples of the esters of carboxylic acids with monohydric alcohols include esters of saturated linear fatty acids having 4 to 22 carbon atoms with branched alcohols having 3 to 22 carbon atoms; branched, saturated fatty acids having 4 to 22 carbon atoms with monohydric alcohols having 2 to 22 carbon atoms; unsaturated fatty acids having 4 to 22 carbon atoms with monohydric alcohols having 2 to 22 carbon atoms; and esters of mono or dicarboxylic acids having 2 to 8 carbon atoms with monohydric alcohols having 2 to 22 carbon atoms.

Examples of the esters with dihydric or higher alcohols include diesters of fatty acids having 4 to 22 carbon atoms with dihydric alcohols having 2 to 6 carbon atoms; diesters or triesters of fatty acids having 4 to 22 carbon atoms with trihydric alcohols having 3 to 6 carbon atoms; and esters of fatty acids having 4 to 22 carbon atoms with tetrahydric or higher alcohols having 6 to 22 carbon atoms.

Specific examples of the esters of the saturated linear fatty acids having 4 to 22 carbon atoms with the branched alcohols having 3 to 22 carbon atoms can include isopropyl octanoate, isopropyl caprinate, isopropyl stearate, isostearyl stearate, isooctyl palmitate, octyldodecyl palmitate, isodecyl myristate, isobutyl myristate, and hexyldecyl myristate.

Specific examples of the esters of the branched, saturated fatty acids having 4 to 22 carbon atoms with the monohydric alcohols having 1 to 22 carbon atoms can include ethyl isostearate, cetyl isostearate, octyldodecyl isostearate, batyl isostearate, isocetyl 2-ethylhexanoate, cetyl 2-ethylhexaoate, cetostearyl 2-ethylhexanoate, and 2-octyldodecyl pivarate.

Specific examples of the esters of the unsaturated fatty acids having 4 to 22 carbon atoms with the monovalent alcohols having 1 to 22 carbon atoms can include octyldodecyl erucate, isodecyl oleate, oleyl oleate, phytostearyl oleate, octyldodecyl ricinoleate, cetyl ricinoleate, and stearyl linoleate.

Specific examples of the esters of the mono or dicarboxylic acids having 2 to 8 carbon atoms with the monohydric alcohols having 1 to 22 carbon atoms can include cetyl lactate, diisooctyl adipate, diisocetyl adipate, dihexyldecyl adipate, diheptylundecyl adipate, di(2-ethylhexyl) succinate, di(ethoxyethyl) succinate, dioctyl sebacate, and dibutyl sebacate.

Specific examples of the diesters of the fatty acids having 4 to 22 carbon atoms with the dihydric alcohols having 2 to 6 carbon atoms can include neopentyl glycol di(2-ethylhexanoate), propylene glycol diisostearate, ethylene glycol dioctanoate, neopentyl glycol dioctanoate, ethylene glycol dioleate, propylene glycol dioleate, ethylene glycol dicaprinate, propylene glycol dicaprinate, propylene glycol dicaprylate, propylene glycol dicaproate, ethylene glycol distearate, diethylene glycol distearate, triethylene glycol distearate, and propylene glycol distearate. Particularly preferred are diesters of fatty acids having 4 to 22 carbon atoms with dihydric alcohols having 2 carbon atoms, for example, ethylene glycol dioctanoate, ethylene glycol dioleate, and ethylene glycol distearate.

Specific examples of the diesters of the fatty acids having 4 to 22 carbon atoms with the trihydric alcohols having 3 to 6 carbon atoms can include glyceryl diisostearate, glyceryl diisopalmitate, glyceryl dioxystearate, glyceryl dioleate, glyceryl distearate, glyceryl dipalmitate, glyceryl dipalmitoleate, glyceryl dibehenate, glyceryl dimyristate, glyceryl di(coconut oil fatty acid), glyceryl dilaurate, glyceryl stearate citrate, glyceryl stearate succinate, glyceryl stearate lactate, and glyceryl stearate malate.

Specific examples of the triesters of the fatty acids having 4 to 22 carbon atoms with the trihydric alcohols having 3 to 6 carbon atoms can include glyceryl triarachidate, glyceryl triisostearate, glyceryl triisopalmitate, glyceryl triundecylate, glyceryl tri(2-ethylhexanoate), glyceryl trierucate, glyceryl trioxystearate, glyceryl trioleate, glyceryl tricaprinate, glyceryl tri(caprylate/caprate/stearate), glyceryl tri(caprylate/caprate/myristate/stearate), glyceryl tri(caprylate/caprate), glyceryl tristearate, glyceryl tri(rapeseed oil fatty acid), glyceryl tripalmitate, glyceryl tripalmitoleate, glyceryl triheptylundecanoate, glyceryl tribehenate, glyceryl trimyristate, glyceryl tri(cottonseed oil fatty acid), glyceryl tri(coconut oil fatty acid), glyceryl trilaurate, glyceryl tri(lanolin fatty acid), glyceryl tri(ricinoleate), glyceryl tri(ricinoleate/caproate/caprylate/caprate), and glyceryl trilinoleate.

Specific examples of the esters of the fatty acids having 4 to 22 carbon atoms with the tetrahydric or higher alcohols having 6 to 22 carbon atoms can include polyglyceryl diisostearate, polyethylene glycol diisostearate, polyethylene glycol dioleate, polyethylene glycol distearate, polyglyceryl distearate, polyglyceryl tetraisostearate, polyglyceryl triisostearate, and polyglyceryl monoisostearate.

Among these, preferred are esters of mono or dicarboxylic acids having 2 to 8 carbon atoms with monohydric alcohols having 1 to 22 carbon atoms; diesters of fatty acids having 4 to 22 carbon atoms with dihydric alcohols having 2 carbon atoms; and triesters of fatty acids having 4 to 22 carbon atoms and trihydric alcohols having 3 to 6 carbon atoms.

Of these, particularly preferred from the standpoint of feeling of use are esters of dicarboxylic acids having 2 to 8 carbon atoms with monohydric alcohols having 8 to 22 carbon atoms, such as diisooctyl adipate and di(2-ethylhexyl) succinate; diesters of fatty acids having 4 to 22 carbon atoms with dihydric alcohols having 2 carbon atoms, such as ethylene glycol dioctanoate, ethylene glycol dicaprate, and ethylene glycol distearate; and triesters of fatty acids having 4 to 22 carbon atoms with trihydric alcohols having 3 to 6 carbon atoms, such as glyceryl trioleate.

Illustrative vegetable oils of the ingredient (B) include olive oil, soybean oil, sesame oil, rapeseed oil, rice bran oil, rice germ oil, wheat germ oil, palm oil, linseed oil, avocado oil, persic oil, carrot oil, kukui nut oil, grapeseed oil, sasanqua oil, safflower oil, almond oil, corn oil, adlay oil, sunflower oil, hazelnut oil, macademia nut oil, meadowfoam seed oil, rose hips oil, peanut oil, coconut oil, castor oil, evening primrose oil, hydrogenated castor oil, jojoba oil, cottonseed oil, and camellia oil, with macademia nut oil, sunflower oil and coconut oil being preferred.

As the ingredient (B), one or more of the above-described oils can be used, and further, the above-described esters and the above-described vegetable oils can also be used in combination. The content of the ingredient (B) ranges preferably from 0.1 to 50 wt. %, more preferably from 0.5 to 30 wt. %, particularly preferably from 1.0 to 10 wt. % based on the cosmetic from the standpoint of whitening effect and feeling of use.

The whitening cosmetic according to the present invention can contain, in addition to the above-described ingredients, one or more ingredients commonly used in cosmetics, quasi-drug products, pharmaceuticals and the like, for example, water, ethanol, oil ingredients other than the ingredient (B), humectants, viscosity increasing agents, preservatives, emulsifiers, medicinal ingredients, powders, colorants, flavors, emulsion stabilizers, pH adjusting agent and the like, as desired.

The whitening cosmetic according to the present invention can be produced in a manner known per se in the art.

The cosmetic according to the present invention is not limited to general skin cosmetics, but includes quasi-drug products, pharmaceuticals for external applications, and the like. A desired preparation form and shape can be chosen depending on the application purpose. For example, it can be formulated into preparation forms such as creams, ointments, milk lotions, lotions, solutions and gels, and into shapes such as packs, sheets, powders and sticks.

### EXAMPLES

### Examples 1-6& Comparative Example 1

Creams shown in Table 2 were produced and were evaluated with regard to whitening effect and feeling of use. The results are also shown in Table 2.

(Production procedure)

Each cream was obtained by heating and melting at 80° C. the corresponding oil-phase ingredients shown in Table 2, adding under stirring the corresponding water-phase ingredients which had been heated to 80° C., and then allowing the resultant mixture to cool down to room temperature under stirring.

(Evaluation method)

(1) Whitening effect

Onto upper branchial parts of 20 normal male volunteers, ultraviolet rays in the UV-B range were irradiated at a dose twice as much as the minimum erythema dose (2MED) once a day for 2 days by using “FS-20SE Lamp” (trade name, manufactured by Toshiba Corporation). Subsequently, the cream was continuously applied to a test area twice a day for 1 month. A measurement was performed with a color-difference meter, and from a Munsell value so obtained, an L* value was calculated. A ΔΔL* value, which is a difference between a change in L* value at the area applied with the cream and a corresponding change at an area not applied with the cream, was determined in accordance with the below-described formula, and was recorded as an index of whitening effect. The results were scored in accordance with the grading scale shown in Table 1, and were indicated in terms of an average score of the twenty volunteers.
ΔΔL*=(L*₁−L*₀)−(L*₁′−L*₀′)
L*₀: Initial value at the area applied with the cream.
L*₀′: Initial value at the area not applied with the cream.
L*₁: Value measured 1 month later at the area applied with the cream.

L*₁′: Value measured 1 month later at the area not applied with the cream.

TABLE 1

| Score | Grading scale |
|---|---|
| 5 | 1.0 ≦ ΔΔL* |
| 4 | 0.6 ≦ ΔΔL* < 1.0 |
| 3 | 0.2 ≦ ΔΔL* < 0.6 |
| 2 | 0 ≦ ΔΔL* < 0.2 |
| 1 | ΔΔL* < 0 |

(2) Feeling of use

By 20 expert panelists, feeling of use upon application of each cream was organoleptically evaluated. The results are shown in terms of the percentage (%) of panelists who answered “good” in the feeling of use (the feeling getting fit to the skin).

TABLE 2

| | | | | | | | Comp. |
|---|---|---|---|---|---|---|---|
| | Example | | | | | | Ex. |
| Ingredient (wt. %) | 1 | 2 | 3 | 4 | 5 | 6 | 1 |
| Oil-soluble chamomilla extract* | 1.00 | 1.00 | 1.00 | 1.00 | 1.00 | 1.00 | 1.00 |
| Ethylene glycol dioctanoate | 3.00 | | | | | | |
| Di(2-ethylhexyl) succinate | | 3.00 | | | | | |
| Diisooctyl adipate | | | 3.00 | | | | |
| Macademia nut oil | | | | 3.00 | | | |
| Coconut oil | | | | | 3.00 | | |
| Sunflower oil | | | | | | 3.00 | |
| POE (60EO) hydrogenated castor oil | 0.50 | 0.50 | 0.50 | 0.50 | 0.50 | 0.50 | 0.50 |
| Glycerin | 0.50 | 0.50 | 0.50 | 0.50 | 0.50 | 0.50 | 0.50 |
| Methylparaben | 0.20 | 0.20 | 0.20 | 0.20 | 0.20 | 0.20 | 0.20 |
| Acrylic acid-alkyl methacrylate | 0.30 | 0.30 | 0.30 | 0.30 | 0.30 | 0.30 | 0.30 |
| copolymer | | | | | | | |
| Potassium hydroxide | 0.16 | 0.16 | 0.16 | 0.16 | 0.16 | 0.16 | 0.16 |
| Ethanol | 5.00 | 5.00 | 5.00 | 5.00 | 5.00 | 5.00 | 5.00 |
| Deionized water | Balance | Balance | Balance | Balance | Balance | Balance | Balance |
| Whitening effect | 4.5 | 4.5 | 4.6 | 4.7 | 4.5 | 4.5 | 3.2 |
| Feeling of use (%) | 85 | 75 | 80 | 85 | 75 | 75 | 25 |

Squalane extract; the content of the spiro ether compound: 250 ppm

From the results of Table 2, the creams according to the present invention all provided good feeling of use and were excellent in whitening effect.

### Example 7

### (Milk Lotion)

A milk lotion having the below-described composition was produced according to a conventional method.

| (Ingredients) | (wt. %) |
|---|---|
| Oil-soluble chamomilla extract | 3.0 |
| (Squalane extract, the content of | |
| the spiro ether compound: 250 ppm) | |
| Ethylene glycol dioctanoate | 3.0 |
| POE sorbitan monostearate | 1.0 |
| Sorbitan monostearate | 0.5 |
| Stearyl alcohol | 0.4 |
| Cetyl alcohol | 0.6 |
| 1,3-Butanediol | 3.0 |
| Glycerin | 1.0 |
| Sodium hyaluronate | 0.5 |
| Tetrasodium edetate | 0.1 |
| Sodium citrate | 0.5 |
| Flavor | q.s. |
| Preservative | q.s. |
| Deionized water | Balance |

### Example 8

### (Essence)

An essence having the below-described composition was produced according to a conventional method.

| (Ingredients) | (wt. %) |
|---|---|
| Oil-soluble chamomilla extract | 0.2 |
| (Squalane extract, the content of | |
| the spiro ether compound: 250 ppm) | |
| Ethylene glycol dicaprate | 1.0 |
| 1,3-Butanediol | 5.0 |
| Glycerin | 4.0 |
| Carboxyvinyl polymer | 0.1 |
| (“Carbopol 981”, trade name; | |
| product of B F Goodrich Co.) | |
| Acrylic acid-alkyl methacrylate copolymer | 0.2 |
| (“Pemulen TR-2”, trade name; | |
| product of B F Goodrich Co.) | |
| L-Arginine | 0.3 |
| Xanthan gum | 0.1 |
| Ethanol | 3.0 |
| Flavor | q.s. |
| Preservative | q.s. |
| Deionized water | Balance |

### Example 9

### (Lotion)

A lotion having the below-described composition was produced according to a conventional method.

| (Ingredients) | (wt. %) |
|---|---|
| Oil-soluble chamomilla extract | 0.1 |
| (Squalane extract, the content of | |
| the spiro ether compound: 250 ppm) | |
| Diisooctyl adipate | 0.3 |
| 1,3-Butanediol | 8.0 |
| Glycerin | 4.0 |
| Sodium hyaluronate | 0.1 |
| Ethanol | 3.0 |
| POE decyl tetradecyl ether | 1.0 |
| Sodium citrate | 0.1 |
| Tetrasodium edetate | 0.1 |
| Flavor | q.s. |
| Preservative | q.s. |
| Deionized water | Balance |

### Example 10

### (Face pack)

A face pack having the below-described composition was produced according to a conventional method.

| (Ingredients) | (wt. %) |
|---|---|
| Oil-soluble chamomilla extract | 1.0 |
| (Squalane extract, the content of | |
| the spiro ether compound: 250 ppm) | |
| Di(2-ethylhexyl) succinate | 0.5 |
| Polyethylene glycol | 3.0 |
| 1,3-Butanediol | 1.0 |
| Glycerin | 2.0 |
| Sodium citrate | 1.0 |
| Disodium edetate | 0.1 |
| Lactic acid | 0.5 |
| Polyvinyl alcohol | 12.0 |
| POE POP decyl tetradecyl ether | 0.3 |
| Preservative | q.s. |
| Flavor | q.s. |
| Deionized water | Balance |

### Example 11

### (Stick)

A stick having the below-described composition was produced according to a conventional method.

| (Ingredients) | (wt. %) |
|---|---|
| Oil-soluble chamomilla extract | 5.0 |
| (Squalane extract, the content of | |
| the spiro ether compound: 250 ppm) | |
| Glyceryl trioleate | 7.4 |
| 2-Ethylhexyl 4-methoxycinnamate | 3.0 |
| Microcrystalline wax | 5.0 |
| Polyethylene wax | 10.0 |
| Particulate zinc oxide | 5.0 |
| Titanium oxide | 10.0 |
| Sericite | 30.0 |
| Mica | 10.0 |
| Kaolin | 5.0 |
| Red iron oxide | 2.0 |
| Yellow iron oxide | 2.5 |
| Black iron oxide | 0.1 |
| POE hydrogenated castor oil | 5.0 |
| (“Nikkol HCO-10”, trade name; | |
| product of Nikko Chemicals Co., Ltd.) | |

### Example 12

### (W/O milk lotion)

A w/o milk lotion having the below-described composition was produced according to a conventional method.

| (Ingredients) | (wt. %) |
|---|---|
| Oil-soluble chamomilla extract | 2.0 |
| (Squalane extract, the content of | |
| the spiro ether compound: 250 ppm) | |
| Ethylene glycol distearate | 1.0 |
| 2-Ethylhexyl 4-methoxycinnamate | 5.0 |
| Particulate zinc oxide | 10.0 |
| Titanium oxide | 20.0 |
| POE-methylpolysiloxane copolymer | 1.0 |
| Methylpolysiloxane | 20.0 |
| Methylcyclopolysiloxane | 20.0 |
| Glycerin | 2.0 |
| Ethanol | 5.0 |
| Preservative | q.s. |
| Flavor | q.s. |
| Deionized water | Balance |

### Example 13

### (Milk lotion)

A milk lotion having the below-described composition was produced according to a conventional method.

| (Ingredients) | (wt. %) |
|---|---|
| Oil-soluble chamomilla extract | 3.0 |
| (Squalane extract, the content of | |
| the spiro ether compound: 250 ppm) | |
| Soybean oil | 3.0 |
| Coconut oil | 3.0 |
| POE sorbitan monostearate | 1.0 |
| Sorbitan monostearate | 0.5 |
| Stearyl alcohol | 0.4 |
| Cetyl alcohol | 0.6 |
| 1,3-Butanediol | 3.0 |
| Glycerin | 1.0 |
| Sodium hyaluronate | 0.5 |
| Tetrasodium edetate | 0.1 |
| Sodium citrate | 0.5 |
| Flavor | q.s. |
| Preservative | q.s. |
| Deionized water | Balance |

### Example 14

### (Essence)

An essence having the below-described composition was produced according to a conventional method.

| (Ingredients) | (wt. %) |
|---|---|
| Oil-soluble chamomilla extract | 0.2 |
| (Squalane extract, the content of | |
| the spiro ether compound: 250 ppm) | |
| Macademia nut oil | 1.0 |
| Coconut oil | 1.0 |
| 1,3-Butanediol | 5.0 |
| Glycerin | 4.0 |
| Carboxyvinyl polymer | 0.1 |
| (“Carbopol 981”, trade name; | |
| product of B F Goodrich Co.) | |
| Acrylic acid/alkyl methacrylate copolymer | 0.2 |
| (“Pemulen TR-2”, trade name; | |
| product of B F Goodrich Co.) | |
| L-Arginine | 0.3 |
| Xanthan gum | 0.1 |
| Ethanol | 3.0 |
| Flavor | q.s. |
| Preservative | q.s. |
| Deionized water | Balance |

### Example 15

### (Lotion)

A lotion having the below-described composition was produced according to a conventional method.

| (Ingredients) | (wt. %) |
|---|---|
| Oil-soluble chamomilla extract | 0.1 |
| (Squalane extract, the content of | |
| the spiro ether compound: 250 ppm) | |
| Sunflower oil | 0.2 |
| Corn oil | 0.2 |
| 1,3-Butanediol | 8.0 |
| Glycerin | 4.0 |
| Sodium hyaluronate | 0.1 |
| Ethanol | 3.0 |
| POE decyl tetradecyl ether | 1.0 |
| Sodium citrate | 0.1 |
| Tetrasodium edetate | 0.1 |
| Flavor | q.s. |
| Preservative | q.s. |
| Deionized water | Balance |

### Example 16

### (Face pack)

A face pack having the below-described composition was produced according to a conventional method.

| (Ingredients) | (wt. %) |
|---|---|
| Oil-soluble chamomilla extract | 1.0 |
| (Squalane extract, the content of | |
| the spiro ether compound: 250 ppm) | |
| Macademia nut oil | 0.5 |
| Avocado oil | 0.5 |
| Polyethylene glycol | 3.0 |
| 1,3-Butanediol | 1.0 |
| Glycerin | 2.0 |
| Sodium citrate | 1.0 |
| Disodium edetate | 0.1 |
| Lactic acid | 0.5 |
| Polyvinyl alcohol | 12.0 |
| POE POP decyl tetradecyl ether | 0.3 |
| Preservative | q.s. |
| Flavor | q.s. |
| Deionized water | Balance |

### Example 17

### (Stick)

A stick having the below-described composition was produced according to a conventional method.

| (Ingredients) | (wt. %) |
|---|---|
| Oil-soluble chamomilla extract | 5.0 |
| (Squalane extract, the content of | |
| the spiro ether compound: 250 ppm) | |
| Grapeseed oil | 2.4 |
| Sunflower oil | 5.0 |
| 2-Ethylhexyl 4-methoxycinnamate | 3.0 |
| Microcrystalline wax | 5.0 |
| Polyethylene wax | 10.0 |
| Particulate zinc oxide | 5.0 |
| Titanium oxide | 10.0 |
| Sericite | 30.0 |
| Mica | 10.0 |
| Kaolin | 5.0 |
| Red iron oxide | 2.0 |
| Yellow iron oxide | 2.5 |
| Black iron oxide | 0.1 |
| POE hydrogenated castor oil | 5.0 |
| (“Nikkol HCO-10”, trade name; | |
| product of Nikko Chemicals Co., Ltd.) | |

### Example 18

### (W/O milk lotion)

A w/o milk lotion having the below-described composition was produced according to a conventional method.

| (Ingredients) | (wt. %) |
|---|---|
| Oil-soluble chamomilla extract | 2.0 |
| (Squalane extract, the content of | |
| the spiro ether compound: 250 ppm) | |
| Safflower oil | 1.0 |
| Sunflower oil | 1.0 |
| 2-Ethylhexyl 4-methoxycinnamate | 5.0 |
| Particulate zinc oxide | 10.0 |
| Titanium oxide | 20.0 |
| POE-methylpolysiloxane copolymer | 1.0 |
| Methylpolysiloxane | 20.0 |
| Methylcyclopolysiloxane | 20.0 |
| Glycerin | 2.0 |
| Ethanol | 5.0 |
| Preservative | q.s. |
| Flavor | q.s. |
| Deionized water | Balance |

All the cosmetics obtained in Examples 7 through 18 provided good feeling of use, were excellent in whitening effect, and were confirmed to be effective for preventing formation of spots and freckles.

### INDUSTRIAL APPLICABILITY

Cosmetics according to the present invention provide good feeling of use upon application, and moreover, are excellent in skin-whitening effect and can satisfactorily prevent formation of spots and freckles which result from sunburn.

1. A whitening cosmetic comprising the following ingredients (A) and (B):
(A) an oil-soluble chamomilla extract, and
(B) at least one oil selected from esters of carboxylic acids having 2 to 22 carbon atoms with alcohols having 1 to 22 carbon atoms and vegetable oils.

2. A whitening cosmetic according to claim 1, wherein the ingredient (A) is a chamomilla extract obtained by extraction with squalane.

3. A whitening cosmetic according to claim 1, wherein in said esters contained as the ingredient (B), said carboxylic acids are fatty acids having 4 to 22 carbon atoms or dicarboxylic acids having 2 to 8 carbon atoms.

4. A whitening cosmetic according to claim 1, wherein in said esters contained as the ingredient (B), said alcohols are monohydric alcohols having 2 to 22 carbon atoms, dihydric alcohols having 2 to 6 carbon atoms or trihydric alcohols having 3 to 6 carbon atoms.
